(19) United States
(12) Patent Application Publication
(10) Pub. No.: US 2007/0112439 A1
(43) Pub. Date: May 17, 2007

(54) Power evacuation valve for prosthetic limb socket

(76) Inventor: Peter V. Panucialman, Melrose Park, IL (US)

Correspondence Address: MAYER, BROWN, ROWE & MAW LLP, P.O. BOX 2828, CHICAGO, IL, 60690-2828, US

(21) Appl. No.: 11/281,637

(22) Filed: Nov. 17, 2005

(51) Int. Cl. A61F 2/74; A61F 2/80

(52) U.S. Cl. 623/26; 623/34

(57) ABSTRACT

A power evacuation valve for a prosthetic limb socket wherein the socket is adapted for direct contact between a patient's skin of a residual limb and an inside surface of the socket, the socket further having an aperture therein for receipt of a valve body of the power evacuation valve, and a method of engaging a stump of a residual limb with an interior of a prosthetic limb socket wherein the prosthetic limb socket has an aperture adapted to receive a valve body of a power evacuation valve.

### BACKGROUND OF THE INVENTION

1. Field Of The Invention

The present invention generally relates to artificial or prosthetic limbs for amputees, and more particularly to a power evacuation valve to assist in removing air from the socket of a prosthetic limb to improve suspension.

2. Discussion of the Prior Art

In the field of fitting amputees with artificial limbs, there are a variety of systems that have evolved over time to enhance retention or suspension of an artificial limb from a residual limb. However, currently there are two types of systems that are most often used. The first, and most widely used, is an artificial limb that has a relatively rigid molded socket that is fitted so as to receive the patient's stump of the residual limb, with direct contact between the socket and the skin of the residual limb. The second, is a more expensive and sophisticated system that requires the patient to use a liner or other insert to cover the stump of the residual limb and incorporates an interface with a relatively rigid molded socket that is configured to develop sub-atmospheric pressures to enhance the integrity of the fit of the artificial limb to the patient's covered stump or specialized insert.

The first type of system is fairly simplistic and cost effective. It does not include a liner or insert, or any other supplemental means of developing sub-atmospheric pressures between the stump and the socket. However, it does have drawbacks. With the first type of prosthetic system, for instance with a lower leg prosthesis, there are several steps required for the patient to achieve insertion into the socket and then attempt to maintain an absence of air between the limb and socket during use.

With the first type of system, the socket typically will have an aperture near the bottom which is configured to receive a plug such as by threaded engagement. To prevent the friction that would otherwise occur when attempting to place the patient's stump into the socket, the patient may use a donning sock. Thus, when using a donning sock to install the artificial limb for use, the patient will start by pulling the donning sock over the stump. Before or after pulling the donning sock over the stump, the patient also will remove the plug from the aperture in the lower portion of the socket. The patient will thread the end of the donning sock through the aperture. Then, the sock covered stump will be inserted into the socket. Next, the patient will grab hold of the donning sock, and pull the donning sock through the aperture while advancing the stump into the socket, thereby leaving the skin of the stump in direct contact with the inner surface of the socket. Finally, the patient will reattach the plug in sealing engagement with the socket. With the stump in the socket and the plug reinstalled, this will cause any attempted removal of the stump from the socket to naturally draw a vacuum within the socket, thereby resisting withdrawal of the stump from the socket.

Unfortunately, without a source of vacuum and without a liner over the stump, it heretofore has not been possible to ensure removal of all of the air between the stump and the socket. Also, it is common for the interface between the skin on the stump and the relatively rigid socket to periodically permit some air to pass by and to enter the socket. When this occurs, the integrity of the fit of the limb and thereby its performance is breached. Indeed, at some point, the suspension will be so inadequate that the patient risks complete detachment from the artificial limb. To reestablish a more secure fit and better suspension, the patient occasionally must force the artificial limb deeper into the socket to reestablish a better fit. This obviously is far from satisfactory performance, may cause some discomfort, and can be very awkward for the patient. To allow the trapped air to escape, the patient must either partially or fully unscrew the threaded plug. Alternatively, the patient may use a plug having a one-way valve. This type of valve typically would be actuated by pressing on a central portion that would allow the trapped air to escape, but would not allow any air to reenter the socket via the valve.

There has been a suggestion that the ease of donning and doffing (or removing a prosthetic limb) may be enhanced by not using a donning sock, but rather by lubricating the skin of the stump or the interior surface of the socket and then using sub-atmosphere pressure to draw the stump into the socket and using positive air pressure to push the stump from the socket for removal. Such a system is disclosed for use in donning and doffing in U.S. Pat. No. 5,658,353. However, the system has drawbacks in that a lubricant must be used which is not only messy and inconvenient, but impractical in that it can be ingested into the pump and cause premature pump failure. Moreover, the modified system also contemplates removal of the suction device, and use of a conventional plug or one-way valve once the prosthetic limb is donned, leading to the same potential problems of compromised engagement and suspension during normal use of the artificial limb. Also, such a system is disclosed as being for use with a standard ac power source, limiting when and where it can be used.

As noted above, the second type of system is more complicated than the first. It is based on achieving and maintaining fairly substantial sub-atmospheric pressures in the socket for an improved fit and suspension. However, such higher sub-atmospheric pressures commonly would be injurious if applied directly to the naked stump. Therefore, the second system employs a liner to cover the stump or an insert device that is used between the stump and the socket, as is disclosed in U.S. Pat. No. 6,726,726 Thus, a liner covered stump or alternative insert device attached to the stump is inserted into a socket, without permitting the skin of the residual limb to be directly exposed to the relatively high sub-atmospheric pressures.

To date, all of the sub-atmospheric systems for improving suspension known to the inventor have been essentially of one of two configurations. The first common form is in a weight activated pump to remove air upon impact, such as when the patient walks or stomps on the ground. These pumps may be referred to as gait-driven pumps, and they can be built into the prosthetic limb, but tend to be heavy and awkward in their manner of use. The second form tends to include an electrically activated pump capable of achieving or, with use of a regulator, maintaining vacuum levels sufficient to ensure a sound fit of a covered stump or insert to the socket. While there are claims that such sub-atmospheric pressures, when used with a stump liner, may serve to enhance circulation and wound healing, the prior art systems capable of sustained sub-atmospheric pressures have required some form of a roll-on urethane or thermoplastic liner or insert to be worn on the stump, to prevent injury to the patient which would occur if the patient's skin is directly subjected to such high levels of vacuum.

These sub-atmospheric type systems have had drawbacks. The need for the liner or insert, and for a power source and pump sufficient to maintain vacuum levels in the range of 10-30 inches of mercury add significantly to the cost of the prosthetic device, and contribute to the weight of the system. Also, these systems are highly customized and generally the entire system must be fabricated and employed together as new equipment. Therefore, these systems do not tend to lend themselves to be adapted or retrofit to the existing artificial limbs used by patients having the above-mentioned first type of system with the relatively rigid molded socket that directly engages the patient's skin.

Accordingly, it is desirable to provide a device that will enhance the fit and performance of prosthetic limb systems, without the additional cost of the stump liner or insert interface, or the weight and size associated with pumps more suited for use in the second type of system. It also is desirable to be able to employ such a device as a retrofit to existing systems of the first type, with portability and attachment to the prosthetic limb to travel with the patient so as to assist in maintaining sound suspension, and without need for messy lubricants, frequent substitution of valves or alternatively the risk of injury to the patient's residual limb due to a more significant sustained use of vacuum. Further, it is desirable to be able to custom design a device into original equipment systems for use by patients that will have skin to socket contact, without significantly complicating the structures involved.

The present invention addresses shortcomings in prior art prosthetic devices, while providing the above mentioned desirable features.

### SUMMARY OF THE INVENTION

The purpose and advantages of the invention will be set forth in and apparent from the description and drawings that follow, as well as will be learned by practice of the invention.

The present invention is generally embodied in a power evacuation valve for a prosthetic limb socket. The power evacuation valve may be embodied in various configurations, with each configuration including a housing having at least two portions. The configurations may include a housing having a valve body connected directly to a first housing portion, so as to locate the entire power evacuation valve assembly adjacent a valve body aperture placed in the socket. Alternatively, the valve body may be remote from the housing, so as to permit the larger housing to be connected to the prosthetic limb at a location spaced from the socket valve body aperture.

In a first aspect of the invention, a power evacuation valve for a prosthetic limb socket is provided for a socket that is adapted for direct contact with the skin of a stump of a residual limb of a patient and that has an aperture in the socket. The power evacuation valve has a housing, a valve body connected to the housing and being adapted to be removably connected to the socket aperture. The power evacuation valve further has an electrical switch connected to the housing, an electrically activated pump, a one-way valve connected in fluid communication with the pump, the valve body being connected in fluid communication with the pump, and at least one battery connected to the electrically activated pump and to the electrical switch.

In another aspect of the invention, the valve body is integrally formed with the housing. In a further aspect of the invention, the valve body is mounted to the housing. In an alternative aspect of the invention, the valve body is spaced from the housing and connected to the housing via a conduit. In a further aspect of the invention, the power evacuation valve includes a regulator to avoid vacuum levels that potentially would injure a patient by automatically preventing the vacuum level from exceeding a preselected sub-atmospheric pressure.

Thus, the present invention presents an alternative to the above-mentioned prior art prosthetic limb socket systems that typically used either no vacuum, or vacuum, positive pressure and messy lubricant during donning and doffing, or such strong sustained vacuum for suspension that a protective liner or insert was required to protect the patient's residual limb from injury. The present invention simplifies the socket to residual limb interface, eliminates the need for a complicated liner or insert, and introduces portability while being readily retrofit to existing sockets of the first type of system.

It is to be understood that both the foregoing general description and the following detailed description are exemplary and provided for purposes of explanation only, and are not restrictive of the invention, as claimed. Further features and objects of the present invention will become more fully apparent in the following description of the preferred embodiments and from the appended claims.

### BRIEF DESCRIPTION OF THE DRAWINGS

In describing the preferred embodiments, reference is made to the accompanying drawing figures wherein like parts have like reference numerals, and wherein:

FIG. 1 is an exploded perspective view, with a socket in cross-section, of a first preferred embodiment of a prosthetic limb having a power evacuation valve.

FIG. 2 is a view of the invention in FIG. 1, but with a patient's stump of a residual limb in the socket and the power evacuation valve connected to the socket.

FIG. 3 is a perspective view of a second preferred embodiment of a power evacuation valve of the present invention, with a housing having an integral valve body with a radial diffuser.

FIG. 4 is a perspective view of a third preferred embodiment of a power evacuation valve of the present invention, with a valve body mounted to a housing and the valve body having a diffuser screen.

FIG. 5 is a fourth preferred embodiment of a prosthetic limb, with a socket in cross-section and having a power evacuation valve housing removably mounted to the socket remotely from a valve body connected to the socket.

FIG. 6 is an exploded perspective view of a valve body for use with a remotely mounted power evacuation valve, such as is shown in FIG. 5.

FIG. 7 is a partially exploded perspective view of a fifth preferred embodiment of a power evacuation valve, with a remote valve body, a rechargeable battery, and first and second housing portions separated for better viewing.

It should be understood that the drawings are not to scale, provide simplified representations of some components, and provide examples of a variety of embodiments that may employ features as desired for the particular application, and are not intended to limit the scope and spirit of the present invention. While considerable mechanical details of a power evacuation valve, including other plan and section views of the particular components, have been omitted, such details are considered well within the comprehension of those skilled in the art in light of the present disclosure. It also should be understood that the present invention is not limited to the preferred embodiments illustrated.

### DETAILED DESCRIPTION OF THE PREFERRED EMBODIMENTS

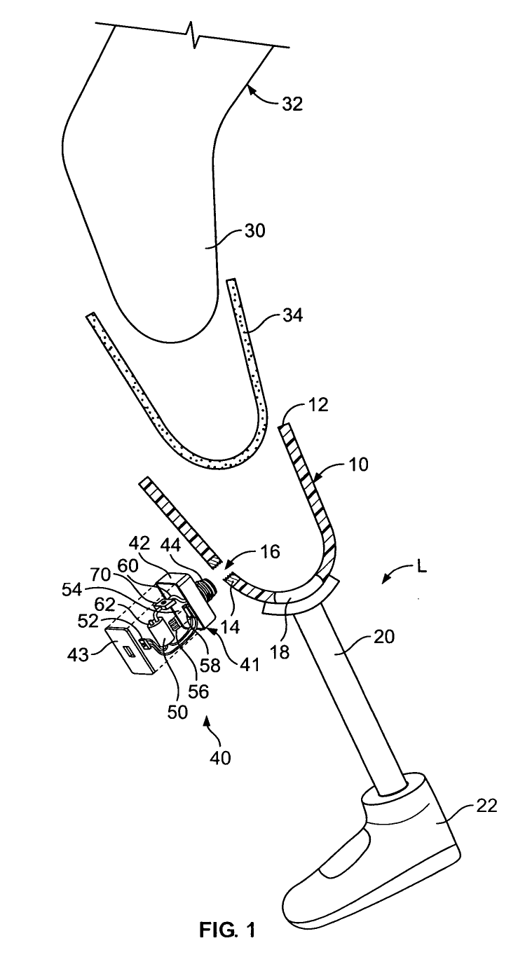

Referring generally to FIGS. 1-7, it will be appreciated that the power evacuation valve of the present invention generally may be embodied within numerous configurations for use with a prosthetic limb socket. Moreover, the invention may be retrofit for use with existing prosthetic limbs having the above-mentioned first type of system.

Referring to a preferred embodiment in FIG. 1, a prosthetic limb L is shown having a formed socket 10 with an upper end 12, and a molded-in-place fitting 14 having a threaded aperture 16. Alternatively, aperture 16 may be formed or cut directly into the material of socket 10. The prosthetic limb L further includes a lower fitting 18 for attachment to a down-tube 20. Mounted at the distal end of down-tube 20 is a foot structure 22. Socket 10 is open at its upper end 12 for receipt of a stump 30 of a residual limb 32 of an amputee patient. It will be appreciated that the power evacuation valve of the present invention may be adapted for use with various artificial limbs, whether for arms, upper legs, lower legs or other uses as needed. Also, while the artificial limb L is shown in a simplified format, it will be understood that any of the many types of artificial limbs may include more sophisticated structures with articulating joints or flexible members as appropriate. Moreover, the materials used in such artificial limbs are not the subject of this invention, and therefore, may be of conventional materials in present use or of any other materials suitable for the particular use.

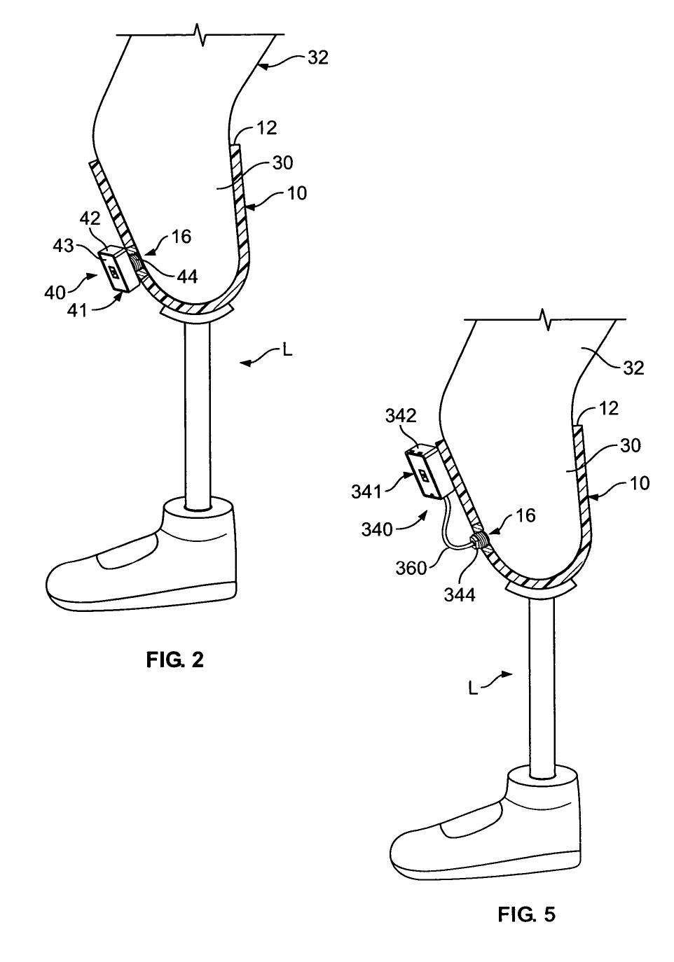

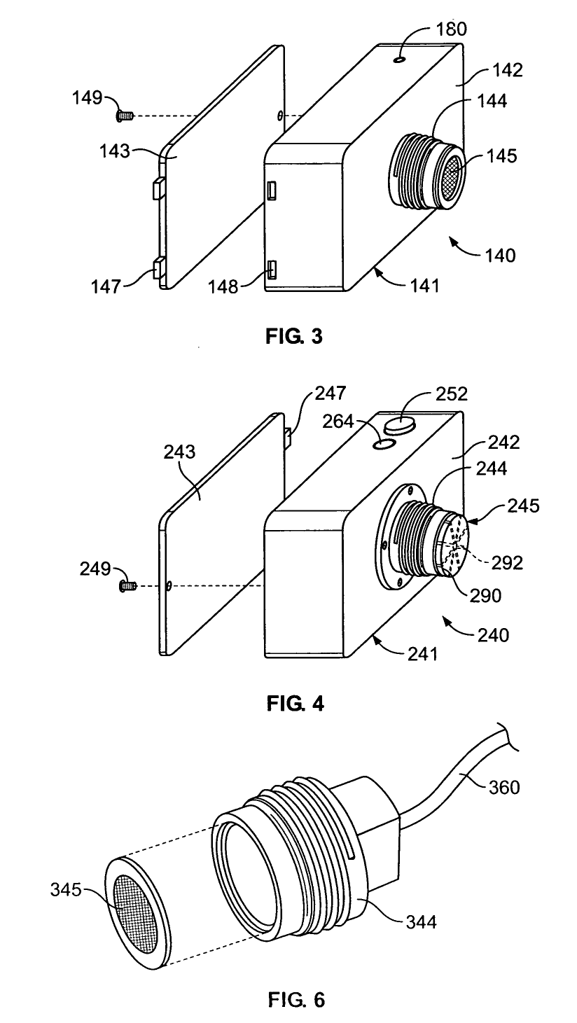

Shown in FIG. 1 is a simplified representation of donning sock 34 for use by a patient to use to cover stump 30 to facilitate insertion of stump 30 into socket 10. A donning sock may be made of a variety of materials and need not necessarily be closed at one end. Also shown in FIG. 1 is a first embodiment of a power evacuation valve 40 of the present invention. Power evacuation valve 40 has a housing 41 shown with a first housing portion 42 and a second housing portion 43. The housing portions may be separate pieces or joined by an integral hinge, and may form a closed body in a variety of ways, such as by snap fit or by fastener(s). A valve body 44 is connected to housing 41 and extends outward therefrom. Valve body 44 (and the other valve bodies disclosed herein) may include an o-ring to achieve a more secure seal to socket 10 when installed. In this embodiment, and the alternative embodiments shown in FIGS. 2 and 3, valve body 44 is formed integrally with first housing portion 42. Also shown in FIG. 1, received within first housing portion 42 is an electric pump 50, electrically connected, such as by wires, to an electrical switch 52 and to a power source 54, shown as a series of batteries 56 in a holder 58. To complete the circuit, the electrical switch 52 also is electrically connected, such as by wires, to power source 54. Electrical switch 52 is connected to second housing portion 43, for ease of activation by the patient.

Electrical pump 50 is shown connected in fluid communication with valve body 44 via a conduit 60. In this embodiment, further connected in line with conduit 60 is a one-way regulator valve 70. Also connected in fluid communication with electrical pump 50 is an exhaust conduit 62. With a housing 41 that is not fully sealed, it is possible to vent or exhaust electrical pump 50 within housing 41 without experiencing back pressure. It will be appreciated that as an alternative to one-way regulator valve 70, a one-way exhaust valve may be used in fluid communication with electrical pump 50, such as at exhaust conduit 62.

To engage stump 30 with socket 10 of prosthetic limb L with the present invention, a patient typically will cover stump 30 with donning sock 34, remove valve body 44 from the aperture 16, and stretch donning sock 34 (or otherwise guide an elongated donning sock) so as to be able to thread an end of donning sock 34 through aperture 16. The patient then will insert sock covered stump 30 through open upper end 12 and into socket 10 while grabbing hold of donning sock 34 to pull it through aperture 16 and remove it from the stump 30, leaving the skin on the surface of stump 30 in direct contact with the inner surface of formed socket 10. After removal of donning sock 34, or alternatively if the patient chooses to insert stump 30 into socket 10 without use of donning sock 34, the patient will then reinstall valve body 44 into aperture 16. This will effectively seal the interior of socket 10 around its periphery via contact with the skin of stump 30.

However, by employing the present invention, the patient may activate electrical switch 52 to energize electrical pump 50 to remove any air trapped between stump 30 and socket 10. Importantly, electrical pump 50 is to be of a type that will draw very low vacuum, ideally of less than 3 inches of mercury, or will be a relatively small pump, for example, Part Number VMP1621CN-06-50 distributed by Virtual Industries, Inc., which runs on 6 volts, and draws 1-10 inches of mercury, or any one of other suitable miniature vacuum pumps such as are available from the same distributor. Such types of pumps may be used in conjunction with a bleeder or regulator valve 70 to prevent the actual vacuum drawn from exceeding a preselected value that is chosen so as not to risk injury to the patient's stump 30, such as 3 inches of mercury or less, or with an automated system that seeks to maintain the vacuum within a preselected range. The battery power required is dependent on the type of electrical pump used, and may be met by use of a series of 3 volt watch batteries, such as model CR2025 Energizer® brand batteries. This low sub-atmospheric pressure helps achieve better engagement between the skin of stump 30 and the interior surface of socket 10 to enhance the retention or suspension of prosthetic limb L, without causing injury to the patient.

In FIG. 2, donning sock 34 has been removed and the skin of the patient's stump 30 of residual limb 32 is in direct contact with the interior surface of socket 1. Stump 30 is held securely in socket 10 of prosthetic limb L by the vacuum produced by power evacuation valve 40 installed in aperture 16.

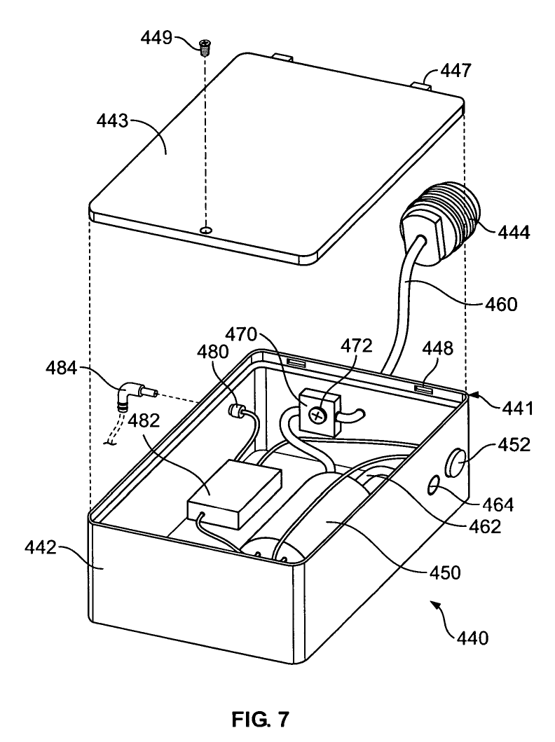

Turning to FIG. 3, an alternative embodiment is shown, where power evacuation valve 140 has a housing 141 with an integrally formed valve body 144. Valve body 144 receives a press-fit diffuser 145. For example, diffuser 145 may be a screen insert having an outer ring and a micromesh screen, which helps reduce the likelihood of injury to the patient by dispersing the suction of the electrical pump over a larger area. In addition, the screen of diffuser 145 helps keep the electrical pump clean by removing contaminants, such as dead skin folicals. First housing portion 142 also is made to accept a second housing portion 143. As mentioned above, it will be appreciated that housing portions may be connected in many ways, one of which is shown in FIG. 3. Second housing portion 143 has tabs 147 that engage slots 148 in an end wall of first housing portion 142, and a threaded fastener 149 is used to engage a threaded aperture (not shown) in first housing portion 142. Also shown in FIG. 3 is a port 180 for receipt of a fitting of an electrical recharger for a rechargeable battery, as will be discussed in more detail in reference to the embodiment shown in FIG. 7.

FIG. 4 illustrates a further alternative embodiment of a power evacuation valve 240. Power evacuation valve 240 is somewhat similar to the embodiment shown in FIG. 3, with a second housing portion 243 that connects to a first housing portion 242 with tabs 247 that engage slots (not shown), and a threaded fastener 249 that engages a threaded aperture (not shown) in first housing portion 242. However, power evacuation valve 240 is shown in an inverted position, with housing 241 end-to-end relative to the view of housing 141 in FIG. 3, and includes alternative structures for second housing portion 242 and valve body 244. As shown, valve body 244 is connected about a peripheral rim to second housing portion 242, and has an alternative diffuser 245. Diffuser 245 has a series of radial channels 290 in fluid communication with a central channel 292 that is in fluid communication with an electrical pump (not shown). Diffuser 245 may be integrally formed with valve body 244 or may be a separate component fitted to valve body 244. Diffuser 245 allows the end of valve body 244 to contact the skin of the patient's stump 30 without the channels 290 being in direct contact with the patient's skin. This structure also permits a single micromesh screen to be used with the central channel 292 if desired, and channel 292 can be enlarged within valve body 244 for use of a large screen component (not shown). Shown in this particular view is an electrical switch 252 and an exhaust port 264, both on a side wall of housing 241, as will be discussed in greater detail in reference to the embodiment shown in FIG. 7.

Turning now to FIG. 5, a further alternative embodiment of a power evacuation valve 340 of the present invention is shown. With this alternative embodiment, a valve body 344 is spaced from a housing 341. This embodiment still permits the valve body 344 to be retrofit into an aperture 16 of an existing socket 10 of a prosthetic limb L, but also allows housing 341 to be placed in a position remote from valve body 344. The valve body 344 is connected via conduit 360 to housing 341, as well as to an electrical pump (not shown) in housing 341. In FIG. 5, housing 341 is shown connected to an exterior surface of socket 10 near its upper end 12. Housing 341 is shown as including a first housing portion 342 and a second housing portion 343, and may be mounted to socket 10 permanently, or may be removably connected to socket 10 such as by a strap, a clamp, a hook and loop fastener system, or the like, for the convenience of the patient. In this view, stump 30 is fully inserted into socket 10 and the skin of stump 30 is already in direct contact with the interior surface of socket 10. With this embodiment, housing 341 may remain in place while valve body 344 is removed to permit insertion of stump 30 into socket 10.

FIG. 6 presents an exploded view of valve body 344, connected to a conduit 360. Valve body 344 may be remote from the housing of the power evacuation valve and preferably has a recess to receive a diffuser 345 that includes a press-fit screen. As with the embodiment shown in FIG. 3, the screen of diffuser 345 of FIG. 6 preferably includes an outer ring and a micromesh screen to disperse the affects of the suction generated while also removing contaminants. It will be appreciated that the diffuser may be embodied in alternative structures and may use alternative filter materials if desired.

Turning to FIG. 7, a further alternative embodiment of a power evacuation valve 440 is shown, with housing 441 having a first housing portion 442 separated from second housing 443. Second housing portion 443 has tabs 447 that engage slots 448 in first housing portion 442, and a fastener 449 to secure second housing portion 443 to first housing portion 442. With second housing portion 443 removed, one can see that switch 452 is electrically connected, such as by wires, to electrical pump 450 and to a suitable rechargeable battery 482. In turn, electrical pump 450 is electrically connected to rechargeable battery 482, such as by wires. Rechargeable battery 482 is chosen in accordance with the power requirements of electrical pump 450, and is further electrically connected to recharger port 480, such as by wires, to permit engagement by a recharger, as illustrated by recharger plug 484.

The embodiment in FIG. 7 has a valve body 444 connected to housing 441 by conduit 460. In addition, valve body 444 is connected in fluid communication with an inlet of electrical pump 450 by conduit 460. Preferably, conduit 460 includes a regulator valve 470 to assist in achieving and maintaining a preselected pressure that will prevent injury to the patient's stump. It will be appreciated that regulator valve 470 may be of any suitable type and preferably is adjustable, such as by adjustment screw 472, or by automated electrical control means (not shown), and preferably incorporates a one-way valve to prevent leakage of air into the socket. The outlet of electrical pump 450 is shown as connected in fluid communication with an exhaust port 464 via conduit 462. It will be understood that conduit 462 may incorporate a regulator and/or one-way valve to achieve the particular design objectives desired.

Thus, it will be appreciated that the present invention can be adapted for use to overcome the disadvantages with existing or previously proposed prosthetic limb systems that either have no supplementary means to achieve sub-atmospheric pressures, or have complicated systems that are messy and inconvenient, or that develop such high sub-atmospheric pressures that they require a protective cover for the patient's stump or some form of an alternative insert to be mounted to the patient's stump for insertion into the socket of a prosthetic limb. The present invention is the first system known to the inventor to be able to be used with existing prosthetic limbs, in place of a simple plug or one-way valve, and which is completely portable so as to travel with the prosthetic limb and provide low vacuum levels that will not injure the patient when exposed directly to the skin of the patient's stump and will enhance suspension of the prosthetic limb. In addition, the invention can be employed in original equipment designs to optimize the size, location and efficiency of the components utilized. In either event, the present invention provides an elegant solution due to its simplicity and ability to be retrofit for immediate use by many, many patients that are not in a position to purchase or be fitted with the more complicated systems that require protective stump covers or socket inserts to be able to withstand the much higher and potentially injurious sub-atmospheric pressures developed in such prior art systems, or that do not want to be hampered by the need to use a lubricant for donning and the insecurity that an adequate engagement and suspension will be maintained after the donning process is completed.

It will be appreciated that a power evacuation valve in accordance with the present invention may be provided in various configurations. Any variety of suitable materials of construction, configurations, shapes and sizes for the components and methods of connecting the components may be utilized to meet the particular needs and requirements of an end user. It will be apparent to those skilled in the art that various modifications can be made in the design and construction of such a power evacuation valve without departing from the scope or spirit of the present invention, and that the claims are not limited to the preferred embodiments illustrated.

What is claimed is:

1. A power evacuation valve for a prosthetic limb socket wherein the socket is adapted for direct contact between a patient's skin of a residual limb and an inside surface of the socket, the socket further having an aperture therein, and the power evacuation valve comprising:
a housing;
a valve body connected to the housing and being adapted to be removably connected to the socket aperture;
an electrical switch;
an electrically activated pump;
a one-way valve connected in fluid communication with the pump;
the valve body connected in fluid communication with the pump;
at least one battery connected to the electrically activated pump and connected to the electrical switch.

2. The power evacuation valve of claim 1, wherein the valve body is integrally formed with the housing.

3. The power evacuation valve of claim 1, wherein the valve body is mounted to the housing.

4. The power evacuation valve of claim 1, wherein the valve body is connected to the housing via a conduit and the housing is adapted to be connected to the prosthetic limb at a location spaced from the valve body.

5. The power evacuation valve of claim 1, wherein the valve body is connected in fluid communication with the pump via a conduit.

6. The power evacuation valve of claim 5, wherein the conduit between the valve body and the pump further comprises a pressure regulator.

7. The power evacuation valve of claim 1, wherein the valve body further comprises a diffuser.

8. The power evacuation valve of claim 7, wherein the valve body diffuser comprises a screen.

9. The power evacuation valve of claim 8, wherein the valve body diffuser screen further comprises a micromesh material.

10. The power evacuation valve of claim 8, wherein the valve body further comprises a recess to accept the screen.

11. The power evacuation valve of claim 7, wherein the valve body diffuser further comprises a plurality of ports in the valve body.

12. The power evacuation valve of claim 1, wherein the at least one battery is rechargeable.

13. The power evacuation valve of claim 1, wherein the pump is vented to the exterior of the housing.

14. The power evacuation valve of claim 1, wherein the pump is vented within the housing.

15. A method of engaging a stump of a residual limb with an interior of a prosthetic limb socket wherein the prosthetic limb socket has an aperture adapted to receive a valve body of a power evacuation valve, the method comprising the steps of:
moving the valve body to a first position in which it will permit air to escape from the interior of the prosthetic limb socket;
inserting the stump of the residual limb into the prosthetic limb socket and permitting direct contact between an interior surface of the prosthetic limb socket and an exterior skin of the stump;
moving the valve body to a second position in which the interior of the prosthetic limb socket is in fluid communication with the power evacuation valve;
activating the power evacuation valve to remove air trapped between the interior surface of the prosthetic limb socket and the skin of the stump so as to achieve a preselected sub-atmospheric pressure; and
keeping the valve body in the second position to permit further activation of the power evacuation valve if the sub-atmospheric pressure fails to be maintained at a preselected value.

16. The method of engaging a stump of a residual limb with an interior of a prosthetic limb socket of claim 15, comprising the further steps of:
covering the stump with a donning sock prior to inserting the stump into the prosthetic limb socket;
passing a portion of the donning sock through the aperture;
inserting the sock covered stump into the prosthetic limb socket;
removing the sock through the aperture in the prosthetic limb socket;
and installing the valve body in the aperture in the prosthetic limb socket prior to activating the power evacuation valve.

17. The method of engaging a stump of a residual limb with an interior of a prosthetic limb socket of claim 15, wherein the valve body is connected to a housing of the power evacuation valve comprising the further step of:
moving the housing of the power evacuation valve with the valve body when moving the valve body to the second position.

18. The method of engaging a stump of a residual limb with an interior of a prosthetic limb socket of claim 15, wherein the valve body is connected to a housing of the power evacuation valve by a conduit, comprising the further step of:
connecting the housing of the power evacuation valve to the prosthetic limb at a location spaced from the valve body.
